(19) United States
(12) Patent Application Publication
(10) Pub. No.: US 2009/0287066 A1
(43) Pub. Date: Nov. 19, 2009

(54) METHOD FOR MINIMALLY INVASIVE MEDICAL INTERVENTION

(76) Inventors: Oliver Meissner, Muenchen (DE); John Christopher Rauch, Savoy, IL (US); Thomas Redel, Poxdorf (DE); Michael Zellerhoff, Forchheim (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/122,856

(22) Filed: May 19, 2008

(51) Int. Cl. A61B 5/00

(52) U.S. Cl. 600/300; 382/128

(57) ABSTRACT

A workflow for a minimally invasive intervention, such as a treatment for a cancerous tumor, includes positioning a patient at a multi-functional imaging apparatus, obtaining pre-interventional images of the anatomy of the patient using a computed tomography or angiography imaging function, performing the minimally invasive intervention while the patient is positioned at the multi-functional imaging apparatus and while using a fluoroscopic imaging function, and performing a post-interventional imaging of the patient's anatomy while the patient is positioned at the multi-functional imaging apparatus using the computed tomography or angiographic imaging function. If the post-interventional imaging determines that additional intervention is in order, the additional intervention is performed while the patient is positioned at the imaging apparatus. Pre-intervention images and data sets from other sources may be combined with or used during the intervention. A treatment planning step may be included following the pre-interventional imaging and the intervention.

### BACKGROUND OF THE INVENTION

1. Field of the Invention

The present invention relates generally to a method for medical intervention for a patient, and more specifically to a method or process as a clinical workflow for minimally invasive therapy utilizing medical imaging of the afflicted area of the patient.

2. Description of the Related Art

Patients who are suspected of having cancer or who have cancerous diseases are increasingly being treated with minimally invasive procedures. The minimally invasive procedures may be performed by interventional radiologists using a percutaneous approach. In the percutaneous approach, a miniature device is inserted into blood vessels or different organ systems, such as the liver, spleen or kidney, through a small opening in the skin and this device is directed to the site of the cancer.

Diagnosis of cancerous diseases may be provided by obtaining a diagnostic biopsy, wherein a sample of tissue from the suspect tissue is obtained and examined by a pathologist. Cancer-killing therapies such as chemotherapy, embolization, radiation, heat and cold, can be delivered directly into the tumor.

Many interventional procedures for the diagnosis and treatment of cancer can be performed on an outpatient basis or during a short hospital stay, without the need for open surgery. In many cases, these procedures offer new cancer treatment options, are less painful and debilitating for the patients, result in quicker recoveries and have fewer side effects and complications. Examples of minimally-invasive interventions that have been successful include radio-frequency ablation (RFA), percutaneous ethanol injection, trans-arterial chemo-embolization, cryo-ablation, laser ablation, microwave ablation, percutaneous brachytherapy, High-intensity focused ultrasound (HIFU) or selective internal radiation therapy (e.g. SIRT, ¹⁸⁸Re-HDD/lipiodol, ¹³¹lipiodol) of liver tumors.

Radio-frequency ablation (RFA) is a technique that uses radio frequency energy via a percutaneous approach to destroy tumor tissue. Selective internal radiation therapy (SIRT) involves the delivery of Yttrium-90 microspheres, beta radiation emitters, into tumor tissue. The microspheres, also known as SIRSpheres, are injected through a transfemoral access into the hepatic artery. The spheres lodge in the small blood vessels of the tumor and irradiate the tumor thus destroying the cancerous tissue while most of the normal liver tissue remains unaffected.

Exact planning of these complex procedures is essential. One aim of the procedures is to focus maximum therapeutic effects inside the region of interest in order to achieve maximum benefit. Another important aim of the procedures is to prevent damage to surrounding tissue. For example, for tumor treatment with thermo-ablative procedures, knowledge of the size of the lesion and the location of adjacent major blood vessels is essential as the relatively cooler blood can absorb some off the heat and carry it away from the site. The loss of heat as a result of the blood flow not only decreases the effectiveness of ablating tissue in the area of interest but also may result in damage to unintended area to which the heated blood is carried. Therefore, functional information such as blood flow or blood volume can add valuable information for treatment planning.

In case of internal radiation therapy, issues related to radiation dosage require detailed knowledge of the total tumor load or the arterial supply of the tumor and the surrounding tissue. Some of this data cannot be obtained using conventional CT or MRI imaging with intravenous contrast injection, but require imaging with selective intra-arterial contrast injection. Selective intra-arterial imaging with contrast requires an intervention consisting of percutaneous arterial access, navigation of a microcatheter to the selected artery of interest, and injection of contrast media during x-ray imaging examination.

With more sophisticated methods for diagnosing and treating cancerous lesions in early stages, demands on imaging modalities increase. Not only are high-end fluoroscopic and cross-sectional imaging required, but there is also a strong demand for real-time functional information. Direct information on tissue perfusion, tumor permeability as well as blood volume and flow rate may allow for immediate therapy control. Moreover, optimal access to the patient, especially in complex situations (patient in intubation anesthesia, patient in prone position) will be helpful for optimal treatment planning.

Currently, there exists no mandatory workflow on how and where to perform minimally-invasive interventions. While most of the punctures, biopsies and therapeutic interventions are performed under CT-guidance, interventions via a vascular access require fluoroscopy/angiography imaging. Moreover, CT is only able to cover a limited field of view, therefore restricting functional information. Additionally, access to the patient during the procedure is limited due to the gantry size of the imaging apparatus.

### SUMMARY OF THE INVENTION

The present invention provides a process as a workflow for a minimally invasive intervention. In particular, a method is provided as a clinical workflow for minimally invasive medical interventions using a an x-ray imaging system, wherein real-time images obtained by a low dose imaging apparatus, for example a fluoroscopic imaging system, of an afflicted area of the patient as obtained during an interventional procedure are used along with high dose images, such as computed tomography images, angiographic images and/or functional images of the afflicted area obtained prior to or following the intervention, or both, without moving the patient from the imaging suite. The patient is positioned at the x-ray imaging apparatus so that the intervention can be performed while using the low dose, real time imaging capability of the imaging apparatus. High dose, pre-interventional images can be obtained of the patient's anatomy while the patient is in position at the imaging apparatus, if desired. Following the intervention, high dose, post-interventional imaging is performed while the patient is still positioned at the imaging apparatus.

In one example, the method includes positioning the patient at the imaging apparatus, performing computed tomography imaging of an afflicted area of the patient possibly including three dimensional reconstruction, cross-sectional imaging and/or functional imaging and analysis, followed by performing the minimally invasive intervention while using fluoroscopic and/or cross-sectional images for guidance during the procedure, and finally performing a further computed tomography imaging following the intervention. As an alternative, the patient is positioned at the imaging apparatus, the minimally invasive intervention is performed while using real-time fluoroscopic imaging of the patient, and then without removing the patient from the imaging apparatus obtaining a follow up image set using higher x-ray doses during imaging and computed tomography, angiography, functional imaging, or other imaging techniques.

### BRIEF DESCRIPTION OF THE DRAWINGS

FIG. 1 is a process flow chart of an embodiment of pre-intervention steps according to the principles of the present invention; and

FIG. 2 is a process flow chart of an embodiment of an intervention and evaluation according to the present workflow.

### DETAILED DESCRIPTION OF THE PREFERRED EMBODIMENTS

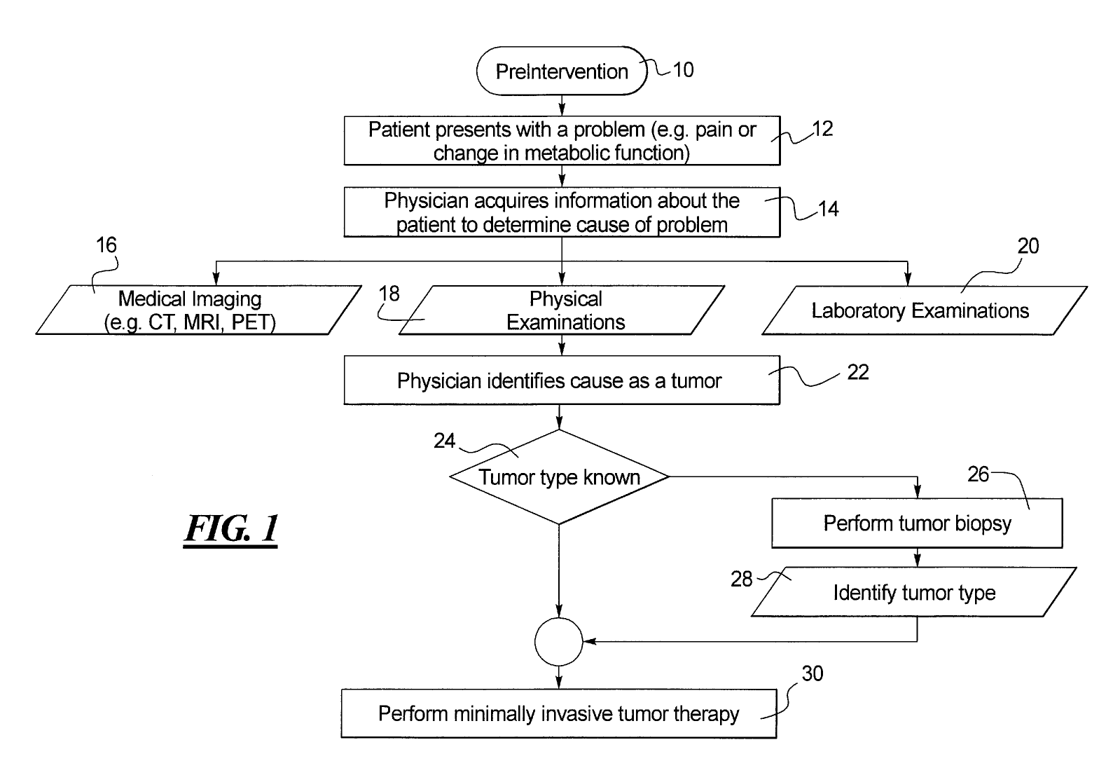

In FIG. 1, workflow process is performed for a patient who in this example is suspected of having a tumor. In the first step, a pre-intervention is begun at 10. The patient presents with a problem, such as a pain or a change in a metabolic function, at step 12. In step 14, the physician acquires information about the patient to determine the cause of the problem. The acquisition of information may include any or all of the steps of performing a medical imaging, such as a CT (computer tomography) imaging, an MRI (magnetic resonance imaging), or a PET (positron emission tomography) imaging, as shown at 16, a physical examination of the patient, at 18, and laboratory examinations or tests, at 20. Any images acquired in this pre-intervention phase are preferably stored for later reference.

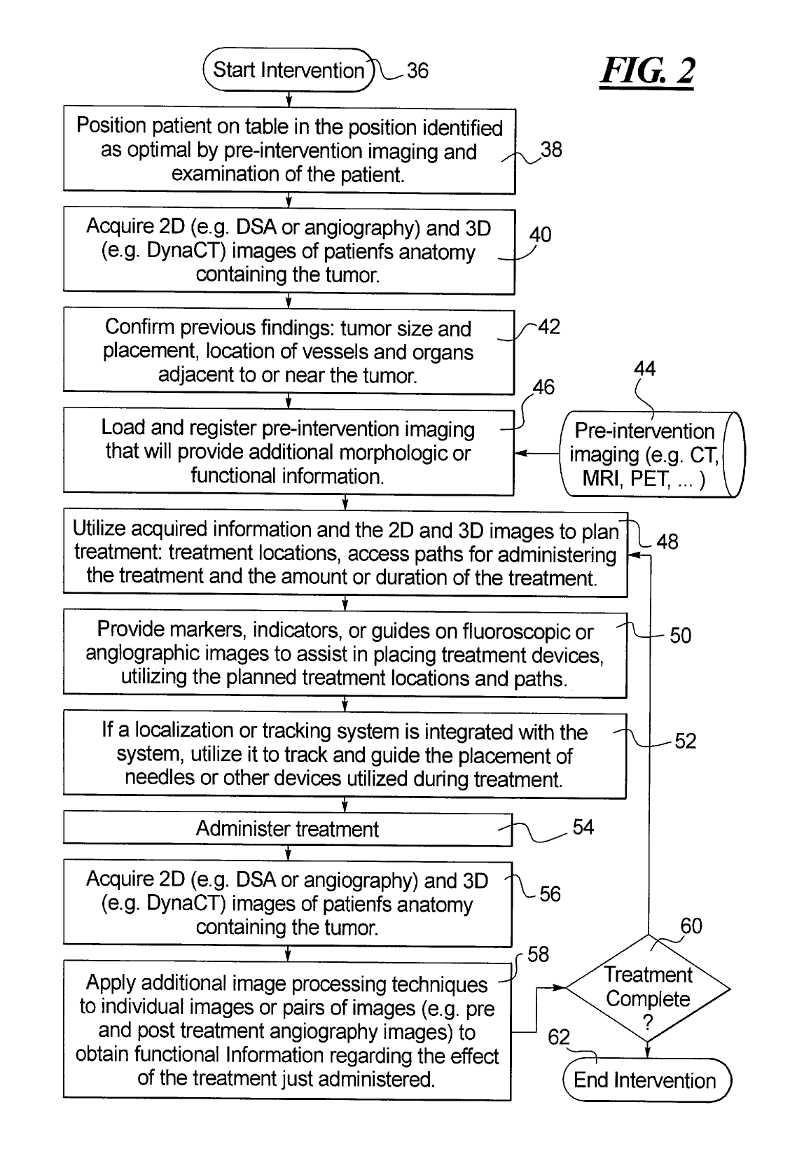

After completing one or more of these investigations, the physician identifies the cause as a tumor, at step 22. At 24, a decision is made as to whether the tumor type is known. If not, a tumor biopsy is performed at 26, possibly using the interventional procedure workflow of FIG. 2, and a determination is made as to the type of tumor, at 28. If the tumor type is known in the decision box 24, a minimally invasive tumor therapy is performed, as indicated at 30. If the tumor type was not initially known and the biopsy has identified the tumor type, the workflow also proceeds to the minimally invasive tumor therapy 30.

Turning now to FIG. 2, a determination has been made to perform a minimally invasive intervention on the patient to treat, remove, or otherwise address the tumor. The workflow begins with a start intervention at 36. The patient is brought to the intervention suite of the medical facility and is positioned on the examination/treatment table in a position that has been identified as optimal by the pre-interventional imaging and the examination of the patient, as indicated at 38. At 40, images of the patient's anatomy containing the tumor are obtained. The images acquired in this step may be two dimensional or three dimensional. Two dimensional images being either fluoroscopic or angiographic and in the form of either native or subtracted images; subtracted images being roadmap fluoroscopy or DSA (digital subtraction angiography) images. Three dimensional images are constructed, using computed tomography techniques, from data acquired by the imaging system during a rotational angiographic acquisition. The 2D and/or 3D images acquired in step 40 are used to confirm any previous findings regarding tumor size and placement, the location of vessels and the location of organs adjacent to or near the tumor, at 42.

Pre-intervention imaging that has been acquired in accordance with the pre-intervention workflow, such as shown in FIG. 1, and have been stored at a storage location 44 as shown in FIG. 2, are loaded at step 46 and registered with the acquired images of step 40. The combination of these images provides additional morphologic and/or functional information for the medical care provider that will be helpful in planning and executing the intervention. At step 48 all of the pre-operatively and intra-operatively acquired images, both 2D and 3D, are utilized to plan the treatment. The treatment planning can include identifying: the treatment locations, the access paths for administering the treatment, and the parameters of the treatment, such as treatment agent amounts, duration of ablations, and device or agent selection. Following planning, treatment may be augmented by the imaging system by the provision of markers, indicators, or guides on either the 2D or 3D images, as shown at 50. These markers and guides are intended to assist in the accurate placement of treatment devices, and in the adherence of the navigation of these devices to the planned treatment paths. If a device localization or device tracking system is integrated with the imaging system, the device localization or device tracking system is used to track and guide the placement of needles or other devices used in the treatment by combining the device location information with acquired 2D and 3D images, as indicated at 52.

An optional step 53 may follow step 52. If a compatible interoperative imaging modality is available (e.g. real-time Ultrasound, endoscopy, IVUS, OCT, etc..) these images can be acquired at any time during the intervention, and if image fusion capability is available the images can be combined with 2D and or 3D images acquired by the x-ray imaging system. Step 54 provides that the treatment or intervention is administered. The treatment or intervention is preferably a minimally invasive intervention. Following the treatment or intervention 54, images are acquired of the patient's anatomy that contains the tumor, as shown at 56. Typically, these images will be acquired using similar, if not identical, imaging methods as performed prior to treatment; the similarly generated images aid in the evaluation of treatment effectiveness. In step 58, additional image processing may be performed on the images individually or collectively, by combining pre-treatment and post-treatment images. This provides the medical care professional with information concerning the effectiveness of the treatment that has just been administered.

Once information is known concerning the effectiveness of the treatment, a decision 60 is made as to whether the treatment is complete. If not, additional treatment or intervention is performed, as indicated by a return to step 48. If so, the intervention is ended at 62.

Thus, the present workflow provides planning, execution and confirmation of the intervention. The execution of the intervention steps and the confirmation of the effectiveness of the intervention are performed without moving the patient from the intervention suite and possibly without moving the patient from their position on the examination and treatment table. Preferably, at least some of the pre-intervention steps, execution of the minimally invasive intervention, and confirmation imaging are all performed without moving the patient from the intervention suite and possibly without moving the patient from the examination table. Of course, all pre-interventional imaging is not performed immediately prior to the intervention, since diagnostic imaging may require study by the medical professionals, the intervention may require informed consent by the patient or the family, treatment of other conditions of the patient, such as lowering blood pressure, treating infection, and the like so there is likely to be lengthy delays between diagnosis and treatment. However, with the present workflow images can be obtained immediately prior to the intervention to detect changes in the patient's condition, changes in the tumor, or other information important to the medical professions to perform the intervention.

By avoiding moving the patient to another room or area of the medical facility between the intervention and confirmation, the discomfort to the patient involved in being moved from place-to-place and in being repositioned at different equipment is eliminated. Further, the time delay between treatment and evaluation is significantly reduced, while also permitting relatively rapid application of a further intervention if the desired effects have not been achieved. In addition, the medical facility does not have schedule separate imaging suites to perform the intervention and the evaluation. Personnel are not required to transport the patient from one place to another. Even more important to the patient's health, if the evaluation determines that a health-effecting complication, such as hemorrhaging, has occurred in the intervention or is occurring as a result of the intervention, the problem is potentially discovered immediately while avoiding the delay of moving the patient to another imaging suite and potentially also addressed immediately rather than after a delay from transport of the patient back to the treatment suite.

The intervention suite of the medical care facility is provided with systems that permit the present workflow to be performed. One such system is an imaging device that combines computed tomography imaging capability with fluoroscopic and angiographic imaging capability. The two dimensional and three dimensional images that are useful in pre-intervention evaluation of the patient and in post-intervention confirmation of the effects of the intervention are obtained with the same device as obtains the fluoroscopic images that are used during the minimally invasive intervention. The patient is positioned once on the examination table and further movement and repositioning of the patient is not required to perform the imaging for the different phases of the workflow.

The combined imaging capability of the device simplifies combining the different image types for the different workflow phases. The images need not be transferred to a network storage location by one imaging system and then retrieved from the storage location by a different imaging system. The images of one type may be maintained in the system for use with, combination with or registration with images of another type obtained by the combination imaging system.

The combined imaging capability as utilized in the present method is provided by devices that offer both computed tomography imaging and fluoroscopy and/or angiography imaging. A C-arm x-ray imaging system is preferred. An example of an apparatus having this capability is the Dyna-CT imaging apparatus (as described in the published application US 2006/0120507, entitled “Angiographic x-ray Diagnostic Device for Rotation Angiography”). With this device, the combination of fluoroscopic/angiographic imaging and cross-sectional imaging becomes possible. With the current systems, however, there still exist some limitations, including for example limited rotational speed of the C-arm, limited degrees of freedom of the system and limited access to the patient.

The present workflow process describes an optimized clinical workflow for minimally-invasive tumor therapy, combining real-time fluoroscopic imaging, cross-sectional imaging and functional imaging in the interventional suite done via a modern C-arm based fluoroscopic and/or angiographic system. In another embodiment, an imaging system enabling such a workflow is based on a multipurpose robotic system, like a system known from EP 0220501, (Kresse, robotic x-ray system). Furthermore, the patent applications, DE 102005012700, “Rontgenvorichtung” and the corresponding application Ser. No. 11/373,698 (not published) describe a robotic angiographic system. However, these disclosures lack any medical workflow, particular for minimally-invasive tumor therapy.

The process workflow for a minimally invasive intervention combines pre-intervention imaging, such as from a computed tomography imaging system, obtained after positioning of the patient at the imaging apparatus with fluoroscopic/angiographic images that are obtained while the patient is as the imaging apparatus to provide availability of detailed two and three dimensional images while also offering real time wide area images during the intervention procedure. Further, detailed post-intervention images are available for evaluating the intervention, without having to move the patient to from the intervention suite.

The workflow is described relative to a minimally invasive intervention for a tumor; however, the present workflow may be used for intervention for a variety of different injuries, ailments and conditions. The intervention may be a minimally invasive intervention or a more conventional surgical intervention.

In another embodiment of the workflow, the clinical workflow includes the following steps:

First, the step of patient positioning is performed. The imaging system typically includes a patient positioning device, for example, a table, on which the patient is positioned. For example, the patient may need to be positioned lying on his or her stomach on the table. In one example, the table on which the patient is positioned is movable, and for example can be tilted by as much as 90 degrees to place the patient in an upright or head-tilt position. The positioning step places the patient for optimal access to the patient by the medical professional and for imaging the desired anatomy by the imaging apparatus, so that depending on the need the patient can be positioned in a supine, prone, upright or side position. The table on which the patient is positioned may be a surgical table, and in most embodiments is a multifunctional table, such as an angiotable. In a specific embodiment, a robotic system is provided to assist in transfer of the patient from a bed or gurney to the table.

The table here may be a bed, patient support platform, chair, backboard, brace or other structure to support, lift, hold, restrain, or otherwise engage the patient in a desired position.

The positioning step has a two fold purpose, namely to position the patient for imaging the anatomy of interest as well as to position the patient for carrying out the intervention. The position of the patient may be the same for both purposes, or the patient may be repositioned between steps or portions of the procedure.

Second, the step of two dimensional and/or three dimensional morphologic and/or functional imaging is performed prior to the intervention. For advanced tumor diagnosis or treatment, the patient will have been positioned on a multipurpose fluoroscopic system for such imaging. A preferred imaging system is a C-arm, cone beam, flat detector imaging system, although other imaging systems are possible. In one embodiment, cross-sectional images are created to permit optimized treatment planning to provide the best possible access to the tumor and for procedure guidance in guiding the treatment devices to the tumor. The cross-sectional imaging allows for detailed information on tumor size, location, and relation to surrounding soft tissue to be determined. To perform the two dimensional and/or three dimensional imaging, one or more rotational computer tomography acquisitions and three dimensional image reconstructions will be performed.

The imaging can be done while using contrast media to enhance aspects of the anatomy within the image, for example the vessels may be highlighted by using intravenous or intra-arterial injection of an iodine based contrast media. In another example, the biliary system may be visualized from an injection of carbon dioxide (CO2) into a biliary duct. Functional imaging and/or analysis, either in two dimensions or in three dimensions, may be performed to achieve advanced information on tumor localization and blood supply, for example to identify areas of increased blood volume and/or blood flow.

The pre-interventional imaging may generate 2D images only, 3D images only, or both 2D and 3D images. The present workflow encompasses use of a variety of different imaging technologies and so any type of medical imaging may be performed within the present workflow.

The image data obtained during the pre-interventional phase may be used alone or may be combined with other previously existing datasets. These datasets may be registered with images acquired by the imaging system, utilizing either 3D to 3D (3D/3D) or 2D to 3D (2D/3D) image registration techniques. This aspect of the workflow is optional and need only be executed if the pre-interventional acquired images contain additional information useful in treatment planning, execution, or evaluation.

It is desirable in some treatments to obtain information on tumor vasculature, i.e. the arterial inflow and venous outflow of the tumor, by performing an angiographic image series (also termed an angio run). The angiographic image series will allow for detailed information on the vascular tree and collateral vessels. Such images require either (selective) intra-arterial or intravenous contrast injection. The pre-interventional images may provide a functional analysis of the 2D angio series as for example the blood flow in arteries near the tumor for advanced planning. Profusion and diffusion measurements may be performed for the tumor and surrounding tissues.

In the third step, treatment planning is performed. The pre-interventional imaging has resulted in providing visibility of the tumor and the surrounding tissues to the medical professional. Using two and/or three dimensional images, the treatment location is determined. The treatment amount or dose to be used during the treatment is determined, for example, by determining the tumor volume, the blood flow to the tumor, and the blood volume in the tumor. These values can be obtained from the images and the imaging dataset. It may be helpful in the treatment planning to determine the organ volume, the blood flow and/or the blood volume of the afflicted organ as well. The health of the organ can be identified from organ and tissue image datasets, so that a determination can be made as to the effect of the treatment on, for example, an impaired organ.

If ablation is to be used during the treatment, the number of ablation steps to be performed is determined, as well as vessel locations and the like. The treatment planning may include path planning of a biopsy needle or other therapeutic devices to reach the tumor, including the determination of the vessels through which the needle or devices will pass and any organs that will be encountered along the path. Tissues to be targeted and tissues to be avoided are identified. Some tissues and organs should be avoided during the treatment and so the planning steps should identify structures that should not be touched.

The structural and functional image data are used for the planning step. Functional information about the anatomy is obtained not just as image data but also as quantitative data obtained from the images. The imaging system thereby plays a critical role in planning the intervention. The functional data, for example, from angiography, may be localized as, for example, a measurement of blood volume. In another embodiment the functional information is used for dosimetric planning. This may include segmentation and volume calculation of the tissue perfused by a selected artery or of the tumors supplied by a selected artery. In larger tumor masses, the information is used for detailed planning of the multiple different therapy locations and options used during the course of the intervention. In an advanced embodiment, the functional information is used for calculation of cooling effects caused by the blood flow in adjacent vessels or inside the tissue being treated.

In step 4, a minimally-invasive interventional procedure is performed. The minimally invasive procedure can include RF, SIRT or other treatments with fluoroscopic and/or cross-sectional guidance using the imaging system on which the patient is positioned. As an optional feature, a real-time 2 d/3 d or 3 d/3 d image overlay can be provided for use for example in puncture guidance. A further option according to some embodiments of the workflow is to provide magnetic tracking in order to place the needle semi-automatically.

The present workflow encompasses any interventional procedure that may be performed while the patient is in the interventional suite.

Step five is generally required but may be an option according to some embodiments and includes post procedural therapy control using fluoroscopic/angiographic imaging, cross-sectional imaging, and/or functional imaging. In other words, an imaging procedure is performed to determine the effectiveness of the treatment. The reasons are two fold, to assess the treatment success and to confirm that no inadvertent harm was done.

In step 6, the intervention is ended, or an additional intervention is performed depending on the results of step five.

Thus, there is provided an optimized clinical workflow for minimally-invasive tumor therapy. The preferred embodiments offer one or more of the following advantages. All 2 d/3 d fluoroscopic, angiographic, cross-sectional and functional information will be available at one time. Decisions can be immediately adapted to the patient. Morphological as well as functional information allows for more sophisticated therapy planning and post-therapeutic success control. Fast and accurate decision-making is possible based on the functional information that is obtained following the intervention. For example, it may be determined whether or not all viable tumor tissue was successfully destroyed. This can directly be transformed into therapy decision.

According to preferred aspects of the workflow, access to the patient is optimized due to the design of the robotic stand on which the patient is positioned and the C-arm imaging system, which is used throughout the workflow.

By providing a dedicated workflow for minimally invasive interventions, errors that may occur do to the complex procedures being performed are reduced.

Thus, there is provided a workflow for a minimally invasive intervention, such as a treatment for a cancerous tumor, includes positioning a patient at a multi-functional imaging apparatus, obtaining pre-interventional images of the anatomy of the patient using a computed tomography or angiography imaging function, performing the minimally invasive intervention while the patient is positioned at the multi-functional imaging apparatus and while using a fluoroscopic imaging function, and performing a post-interventional imaging of the patient's anatomy while the patient is positioned at the multi-functional imaging apparatus using the computed tomography or angiographic imaging function. If the post-interventional imaging determines that additional intervention is in order, the additional intervention is performed while the patient is positioned at the imaging apparatus. Pre-intervention images and data sets from other sources may be combined with or used during the intervention. A treatment planning step may be included following the pre-interventional imaging and the intervention.

Although other modifications and changes may be suggested by those skilled in the art, it is the intention of the inventors to embody within the patent warranted hereon all changes and modifications as reasonably and properly come within the scope of their contribution to the art.

We claim:

1. A workflow method for a minimally invasive interventional procedure, comprising the steps of:
positioning a patient on a multipurpose fluoroscopic imaging device;
obtaining a cross-sectional image of an afflicted area of a patient;
performing a rotational image scan of the afflicted area of the patient;
constructing a three dimensional image of the afflicted area of the patient;
performing a functional imaging and analysis of the afflicted area of the patient;

2. A workflow process for minimally invasive interventional procedure on a patient, comprising the steps of:
bringing a patient to an interventional suite;
positioning the patient in a position relative to an imaging apparatus;
performing pre-interventional imaging of at least a portion of the patient while the patient is in the position;
constructing first images of the portion of the patient for use during the minimally invasive interventional procedure;
planning the minimally invasive interventional procedure using the first images while the patient is in the position;
performing the minimally invasive interventional procedure on the patient using the first images while the patient is in the position;
obtaining second images of the portion of the patient during the minimally invasive intervention;
ending the minimally invasive intervention; and
obtaining third images of the portion of the patient to aid in evaluating the minimally invasive intervention while the patient is in the position.

3. A workflow process as claimed in claim 2, wherein said imaging apparatus is a C-arm imaging apparatus.

4. A workflow process as claimed in claim 2, wherein said first images are obtained at a relatively higher radiation dose than said second images.

5. A workflow process as claimed in claim 2, wherein said step of constructing said first images include constructing three dimensional images of the portion of the patient.

6. A workflow process as claimed in claim 2, wherein said first images include functional images of the portion of the patient.

7. A workflow process as claimed in claim 2, wherein said first images include 2d or 3d angiographic images of the portion of the patient.

8. A workflow process as claimed in claim 2, wherein said first images used for performing the minimally invasive interventional procedure are used for navigation of the devices including catheters and needles.

9. A workflow process as claimed in claim 2, wherein said first images are used for dosimetric planning.

10. A workflow process as claimed in claim 9, wherein dosimetric planning is used for radioactive treatment as SIRT.

11. A workflow process as claimed in claim 2, wherein said first images are used for planning of ablative treatment.

12. A workflow process as claimed in claim 11, wherein said ablative treatment includes RF ablation cryotherapy, thermo ablation. LIF.

13. A workflow process as claimed in claim 11, wherein planning includes calculation of effective ablation zones including thermal diffusion and cooling.

14. A workflow process as claimed in claim 2, further comprising the step of:
registering said first images with said second images for use during said minimally invasive intervention.

15. A workflow process as claimed in claim 2, wherein said second images are fluoroscopic images.

16. A workflow process as claimed in claim 2, wherein said step of constructing said third images include constructing three dimensional images of the portion of the patient.

17. A workflow process as claimed in claim 2, wherein said third images include functional images of the portion of the patient.
